(19) United States
(12) Patent Application Publication
(10) Pub. No.: US 2024/0352451 A1
(43) Pub. Date: Oct. 24, 2024

(54) A METHOD OF LOADING DEVICES USING ELECTROWETTING

(71) Applicant: NUCLERA LTD, Cambridge Cambridgeshire (GB)

(72) Inventors: Michael Jan Horka, Cambridge (GB); Sumit Kalsi, Cambridge (GB)

(21) Appl. No.: 18/578,094

(22) PCT Filed: Jul. 21, 2022
(86) PCT No.: PCT/GB2022/051887

§ 371 Date: Jan. 10, 2024

(30) Foreign Application Priority Data
Jul. 21, 2021 (GB) 2110452.6
Mar. 14, 2022 (GB) 2203522.4

(51) Int. Cl. C12N 15/10; G01N 27/447

(52) U.S. Cl. CPC C12N 15/1068; G01N 27/44756

(57) ABSTRACT

The invention relates to improved methods of loading aqueous reagents into electrowetting devices which are often hydrophobic and therefore problematic to load. Disclosed is a method for moving an aqueous droplet from an inlet port onto an EWoD device by actuating a temporary inlet path.

### FIELD OF THE INVENTION

This invention is in the field of fluid electrokinetics: Electrowetting-on-dielectric (EWoD) and Dielectrophoresis (DEP); and the devices using these phenomena. The invention relates to improved methods of loading aqueous reagents into devices with different surface energies, and internal hydrophobic areas, which otherwise make the loading process difficult.

### BACKGROUND

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c . {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction. The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP.

EWoD uses electric field for manipulation of liquid droplets and to perform droplet operations such as movement, mixing and splitting. The droplets are usually generated from an interstitial reservoir, formed in the cell gap, which is metered in via a fluid applicator e.g. pipette or automated fluid delivery sub-system.

Loading of a metered volume in a bubble free manner is critical. However, due to the presence of low surface energy hydrophobic material used on EWoD, capillary filling of aqueous reagents is energetically unfavourable. Most of the plastic housing on top of these devices have lower contact angle (higher surface energy) and tends to draw the liquid back out of the hydrophobic EWoD to fill the port instead of the internal interstitial reservoir. Moreover, during the process of loading, air bubbles can easily be introduced which block the reservoir filling and affect its function. Pressurising of the liquid in order to force entry often results in the liquid being forced back out of the inlet port when the pressure is released. Some of these actions may also lead to contamination between the neighbouring port reagents.

There are many prior art methods of loading EWoD devices which are sub-optimal. For example US2015352544 describes a system configured to conduct designated reactions for biological or chemical analysis. The system includes a liquid-exchange assembly comprising an assay reservoir for holding a first liquid, a receiving cavity for holding a second liquid that is immiscible with respect to the first liquid, and an exchange port fluidically connecting the assay reservoir and the receiving cavity. The system also includes a pressure activator that is operably coupled to the assay reservoir of the liquid-exchange assembly. The pressure activator is configured to repeatedly exchange the first and second liquids by (a) flowing a designated volume of the first liquid through the exchange port into the receiving cavity and (b) flowing a designated volume of the second liquid through the exchange port into the assay reservoir. The system also includes a fluidic system that is in flow communication with the liquid-exchange assembly.

US2020108396 describes a microfluidic device which comprises upper and lower spaced apart substrates defining a fluid chamber therebetween; an aperture for introducing fluid into the fluid chamber; and a fluid input structure disposed over the upper substrate and having a fluid well for receiving fluid from a fluid applicator inserted into the fluid well. The fluid well communicates with a fluid exit provided in a base of the fluid input structure, the fluid exit being adjacent the aperture. The fluid well comprises first, second and third portions, with the first portion of the well forming a reservoir for a filler fluid; and the second portion of the well being configured to sealingly engage against an outer surface of a fluid applicator inserted into the fluid well. The third portion of the well communicates with the fluid exit and has a diameter at the interface between the third portion and the second portion that is greater than the diameter of the second portion at the interface between the third portion and the second portion.

US20200269249 describes microfluidic devices and loading thereof. The devices can be loaded using various means, including the use of force from a filler fluid or air bubble. Various loading methods are described, including drawing fluid away from the entry point. However such methods do not solve the problem of accurately loading a defined fluid volume without loading bubbles or using force. The publication also describes methods of removing fluid from a device and the use of permanently actuated channel to keep liquids away from certain area of the device where the liquid would be undesired.

For example [0055] recites:
Forcing, or urging, the working fluid from the loading region into the fluid chamber may comprise dispensing a second fluid from the fluid applicator to thereby force the working fluid from the loading region into the fluid chamber via the aperture. In this embodiment the fluid applicator is further actuated to dispense a bubble of air (or other fluid different to the working fluid being dispensed), so as to load the working fluid fully into the desired region of the microfluidic device.
The second fluid may be a fluid different to the working fluid. The second fluid may for example be air, or may be filler fluid.

US2011311980 describes methods for and uses of droplet actuation. The loading of the devices suffers from the problems of loading inaccuracy, introduction of bubbles and ejection from the device when a positive pressure is removed.

It is therefore an object of the invention to provide an improved method for loading aqueous liquid into EWoD devices.

### SUMMARY OF THE INVENTION

Provided herein is a method for the controlled filling of a reservoir on a microfluidic device. More specifically, a method for controlled filling of a reservoir in an electrowetting on dielectric (EWoD) device having hydrophobic surfaces.

Loading of reagent is a function of several inter-dependent processes; EWoD forces to allow (and retain) the reagent in the device; capillary forces which draw the reagent back into the plastic inlet port, fluctuations in the cell gap during a pressure driven loading; surface tension of the reagents and the Laplace pressure defined by the shape of the reagent in the plastic port and the nominal cell gap. Typically interior surfaces of the device are more hydrophobic than the surfaces of the plastic housing of the loading port. The invention relates to improved method for loading multiple aqueous reagents and with a higher accuracy for the target and metered volumes. Therefore, the presented method is especially beneficial for loading reagents with high surface tension into device with a low surface energy.

The invention relates to improved methods of loading aqueous reagents where a net force otherwise results in back flow of fluid after being loaded. The prior art loading methods, for example as described in US20200269249 either use a positive pressure, or introduce air bubbles, or result in inaccurate loading volumes from variable input volumes.

According to the invention there is provided a method for loading aqueous liquids from an external source into a planar EWoD device having an array of electrodes, the method comprising;
a. taking an EWoD device having an inlet port connected to an external source,
b. actuating reservoir electrodes to form a defined reservoir of liquid on the device wherein the defined reservoir is separated from the inlet port by at least two electrodes so as not to overlap the inlet port; and
c. actuating specific path electrodes on the device in the vicinity of the inlet port to form a virtual path for liquid entry from the external source over the electrodes onto the device, wherein the virtual path is narrower than the reservoir.

The method loads the reservoir using a narrow neck of fluid that can be snapped when a portion of the electrodes in the entry path are switched off. Once the fluid neck in the path is snapped, the liquid can no longer flow towards the inlet port. Many reservoirs can be loaded in parallel from many entry ports. The entry path may comprise a path of varying widths along the path, and a subset of the electrodes on the path can be switched off to disconnect the entry path from the inlet port without moving the reservoir away from the inlet port. The inlet path may comprise a perpendicular bar in order to form a cruciform shape. The cruciform shape provides a decreased radius of curvature for the liquid, in order to promote snapping of the liquid when the path electrodes are switched off. The cruciform shape provides a decreased radius of curvature from both sides of the neck of fluid, resulting in increased Laplace pressure which promotes snapping of the liquid when the path electrodes are switched off or the backflow of oil occurs. The electrodes are switched off in the region between the perpendicular bar and the reservoir. Efficient snapping of liquid may be obtained by pulsing the electrodes, such that they turn off, on and off again. The repeated pulsing allows the neck to narrow before snapping, thereby accurately controlling the liquid volume in each reservoir.

Disclosed is a method for loading aqueous liquids from an external source into a planar EWoD device having an array of electrodes, the method comprising;
a. taking an EWoD device having an inlet port containing an aqueous liquid,
b. actuating reservoir electrodes to form a defined reservoir of aqueous liquid on the device wherein the defined reservoir is separated from the inlet port by at least two electrodes so as not to overlap the inlet port;
c. actuating specific path electrodes on the device from the inlet port to form a virtual path for aqueous liquid entry over the electrodes onto the device, wherein the virtual path is narrower than the reservoir; and
d. switching off at least two of the electrodes in the virtual path to separate the reservoir from the inlet port, thereby preventing back-flow of the aqueous liquid from the reservoir to the inlet port.

The actuation time for the virtual path electrodes can be controlled to define the volume of liquid on the device. The liquid on the device can be held in a defined area using electrode activation to form an on-chip reservoir isolated from the inlet port. The internal reservoir is defined by a volume of immobilized liquid. Any extra volume cannot be held by non-activated electrodes and extends beyond the reservoir and is free to diffuse around the EWoD device. Electrode activation allows the otherwise hydrophobic surface to become more hydrophilic.

The electrodes forming the path can remain actuated and the external source of liquid removed from the inlet, thereby breaking the fluid connection between the internal reservoir and the external source. However this may result in ejection of liquid. The method may therefore be performed such that electrodes in the virtual path are switched off in order to prevent back-flow of reagents from the reservoir to the inlet port.

The virtual path is formed by actuating electrodes on the device and the internal aqueous liquid is held in place by electrode actuation to form an internal reservoir. The number of electrodes activated to form the width of the virtual path is less than the width of the defined reservoir. The number of electrodes activated to form the width of the virtual path can be less than half the number forming the width of the defined reservoir. The number of electrodes activated to form the width of the virtual path can be less than a quarter the number forming the width of the defined reservoir. The length of the electrode path must be at least two electrodes in order to generate spatial isolation from the entry inlet. The length of the electrode path must also be greater than the cell height (the gap between the two surfaces of the EWoD device). The virtual path may not be in direct contact with a loading port, which minimizes the influence of capillary effect by immediate pinching of the aqueous bridge with a continuous phase. Alternatively the path may contact the loading port and the path snapped once the reservoir is formed on the device.

In order to help prevent bubble entry, multiple virtual paths can be formed to connect the inlet to the reservoir. Thus multiple virtual paths can connect the inlet to the reservoir, either at the same time or sequentially.

The inlet port can be formed by a hole in the upper surface or side of the planar EWoD device. The hole can be approximately 1 mm in diameter.

The array of electrodes can be formed on the opposing surface of the planar EWoD device to the surface having the entry hole.

The external source can take the form of a pipette or delivery tube. The pipette may be a multi-channel pipette, for example having 4, 8 or 12 channels.

The electrode actuation can occur for a period of greater than 1 second. The electrode actuation can occur for a period of 10-120 seconds.

The delivery path can be formed by actuating greater than 2 electrodes. The delivery path can be formed by actuating between 10-500 electrodes arranged in an elongated pattern. The delivery path can be formed by actuating electrodes arranged in an elongated pattern of 35 long by 8 wide. The delivery path can be formed by actuating electrodes arranged in an elongated pattern of 22 electrodes long by 4 electrodes wide. The pattern can be 22-35 electrodes long and 4-8 wide.

The delivery path defines a path along which liquid enters the reservoir. The path can be switched off along all or a portion of its length in order to snap the aqueous liquid connection between the inlet and the reservoir.

Where the path defines a cruciform shape, the dimensions may be for example:

The reservoir may be 16 pixels wide, where each pixel is one electrode. The width can vary along the length of the path, and is independent of the reservoir. The drawbridge may consist of 4 parts:
1) Connector from the inlet port: 6×10 (width×length)
2) Perpendicular bar: 16×4 (thus can optionally be as wide as the reservoir)
3) Initial neck formation: 4×6
4) Pulsing section: 6×6

Thus the snapping point does not have to be at the narrowest point on the path. In said example, the path has four sections, one of which is deactivated to break the connection. In the example given above the reservoir is 26 electrodes from the inlet (10+4+6+6).

The path may be broken between the reservoir and the perpendicular bar, hence leaving the cruciform shape disconnected from the reservoir. The remainder of the virtual path is generally left activated in order to ensure any residual liquid does not move around on the device.

The on-chip reservoir can be formed 2-500 electrodes away from the inlet port. The on-chip reservoir can be formed 20-100 electrodes away from the inlet port. The on-chip reservoir can be formed with 0.1 to 100 μL. Multiple on-chip reservoirs can be formed using a single inlet port. Alternatively multiple inlet ports can be used to combine reagents into one or more on-chip reservoirs.

Also provided herein is a method for visual indication of drop or reservoir area. The measurement of area requires a change in the shape of the droplet. For instance to measure a large drop of enclosed aqueous liquid on the panel, changing the shape of the enclosed liquid to include protrusions indicates the area of the liquid. For example a square covering say 10×10 pixels (100 pixel area) is difficult to measure an accurate area. A difference of 5% in a square may be difficult to measure. If the liquid covers a linear area of 100 pixels, when the area is 5 pixels too small the difference is easier to detect as the line only covers 95 pixels. Thus the area of long thin shapes is easier to accurately measure than square shapes. Disclosed is a way of accurately measuring the area of an enclosed aqueous fluidic liquid on an electrowetting on dielectric (EWoD) device by dynamically elongating the enclosed liquid to determine the size covered and allowing the enclosed liquid to return to a more compact shape once the area is known. The enclosed aqueous fluidic liquid can be a reservoir from which droplets are dispensed, or dispensed droplets.

The method can be applied to loading a reservoir. By introducing virtual calibration structures on the interstitial reservoirs of the device, a visual signal is produced which assists in the accurate loading of the reservoir with a defined area of liquid. These calibration structures are formed during the loading cycle of reagents and merged into the reservoirs during the dispense cycling. In effect the calibration structures are temporarily actuated electrodes on the far side of the reservoir electrodes from the fluid inlet. Once these temporally transient areas are filled, the electrodes forming the calibration structures are switched off and the liquid joins the main reservoir.

Disclosed herein is a method for dynamically controlling the shape of aqueous phase for the controlled filling of a reservoir on a planar electrowetting on dielectric (EWoD) device, the method comprising:
a. taking an EWoD device having an internal or external reagent source liquid;
b actuating reservoir electrodes to form a defined reservoir of aqueous liquid on the device wherein the defined reservoir is separated from the source liquid by at least two electrodes; and
c. temporarily actuating electrodes on an opposing side of the reservoir to the source liquid to form one or more virtual calibration structures which are the last areas to fill, such that when the temporarily actuated electrodes are switched off the liquid becomes part of the reservoir, thereby accurately controlling the liquid area in the reservoir.

Disclosed is a method for loading aqueous liquids from an external source into a planar EWoD device having an array of electrodes, the method comprising;
a. taking an EWoD device having an inlet port containing an aqueous liquid,
b. actuating reservoir electrodes to form a defined reservoir of aqueous liquid on the device wherein the defined reservoir is separated from the inlet port by at least two electrodes so as not to overlap the inlet port and the reservoir includes electrodes on an opposing side of the reservoir to the source liquid to form one or more virtual calibration structures which are the last areas to fill;
c. actuating specific path electrodes on the device from the inlet port to form a virtual path for aqueous liquid entry over the electrodes onto the device, wherein the virtual path is narrower than the reservoir; and
d. switching off at least two of the electrodes in the virtual path to separate the reservoir from the inlet port, thereby preventing back-flow of the aqueous liquid from the reservoir to the inlet port.

The virtual calibration structures can be switched off at the same time as the path electrodes or subsequent to the path electrodes.

Disclosed is a method for loading aqueous liquids from an external source into a planar EWoD device having an array of electrodes, the method comprising;
a. taking an EWoD device having an inlet port containing an aqueous liquid,
b. actuating reservoir electrodes to form a defined reservoir of aqueous liquid on the device wherein the defined reservoir is separated from the inlet port by at least two electrodes so as not to overlap the inlet port and the reservoir includes electrodes on an opposing side of the reservoir to the source liquid to form one or more virtual calibration structures which are the last areas to fill;
c. actuating specific path electrodes on the device from the inlet port to form a virtual path for aqueous liquid entry over the electrodes onto the device, wherein the virtual path is narrower than the reservoir and forms a cruciform shape;
d. switching off at least two of the electrodes in the virtual path to separate the reservoir from remaining cruciform shape and hence the inlet port, thereby preventing back-flow of the aqueous liquid from the reservoir to the inlet port; and
e. switching off the virtual calibration structures.

The virtual calibration structures can be elongated protrusions. There can be more than one elongated protrusions per reservoir. There can be two or three elongated protrusions per reservoir.

Each of the elongated protrusions may comprise 1 to 10% of the area of the reservoir.

An opposing side may be at 90 degrees or 180 degrees in relation to the source of the flowing liquid entering the reservoir. The virtual calibration structures may be on more than one opposing side from the reservoir entry.

US 2011/0220505 describes a method for transferring liquid on a EWoD device using actuation alone to initiate a flow of liquid from a reservoir. The actuation for entry can be performed using multiple channels (‘toothed electrodes’). The teeth are used to move and steer the liquid. Therefore the protrusions are on the same side as the incoming liquid. The invention as claimed herein using the protrusions on an opposing side to the inlet port in order to accurately control the loading area.

Disclosed is a method wherein the EWoD device includes:
a first substrate having a matrix of electrodes, wherein each of the matrix electrodes is coupled to a thin film transistor, and wherein the matrix electrodes are overcoated with a functional coating comprising:
   a dielectric layer in contact with the matrix electrodes,
   a conformal layer in contact with the dielectric layer, and
   a hydrophobic layer in contact with the conformal layer;
a second substrate comprising a top electrode;
a spacer disposed between the first substrate and the second substrate and defining an electrokinetic workspace; and
a voltage source operatively coupled to the matrix electrodes.

The loading can be performed on aqueous liquids without the need for surfactants. The aqueous liquid can have a substantial ionic strength, for example an ionic strength greater than 0.1 M. The aqueous liquid can include biological reagents, for example nucleotides, enzymes, oligonucleotides or the components for protein expression.

Disclosed is a method for performing droplet based nucleic acid synthesis, wherein the method comprises repeating the methods herein in order to add nucleotides to an initiation oligonucleotide.

Disclosed is a method for performing droplet based nucleic acid assembly, wherein the method comprises repeating the methods herein in order to join two or more nucleic acid strands in one or more droplets.

Disclosed is a method for performing droplet based cell-free expression of peptides or proteins, wherein the method comprises repeating the methods herein wherein the droplets contain nucleic acid templates and a cell-free system having components for protein expression.

### FIGURES

FIG. 1a: Cross sectional view of a planar EWoD based microfluidic device, showing a fluid loading port. The device comprises a polymer housing for interfacing to an external source; a top substrate and a bottom substrate with electrodes which are separated by a spacer to form the cell gap for fluid droplet manipulation. The top substrate has a hole to allow the fluid entry into the cell gap.

FIG. 1b: Cross sectional view of a planar EWoD based microfluidic device, showing a fluid loading port. The device comprises a polymer housing for interfacing to an external source; a top substrate and a bottom substrate with electrodes which are separated by a spacer to form the cell gap for fluid droplet manipulation. The top substrate has an under-hang over the bottom substrate, and forms a channel for fluid to enter the cell gap.

FIG. 2: Top view of a planar EWoD based microfluidic device showing schematic of top and bottom substrates with an entry hole or channel into the cell gap. (A) Top and bottom substrates are aligned with adhesive line forming a cell gap of fixed height, the holes in top substrate align with the loading port in polymer housing to deliver fluid to the cell gap. (B) The top substrate has an under-hang over the bottom substrate, the adhesive line forms channel of entry for fluid into the cell gap. The loading port in polymer housing is aligned with each channel.

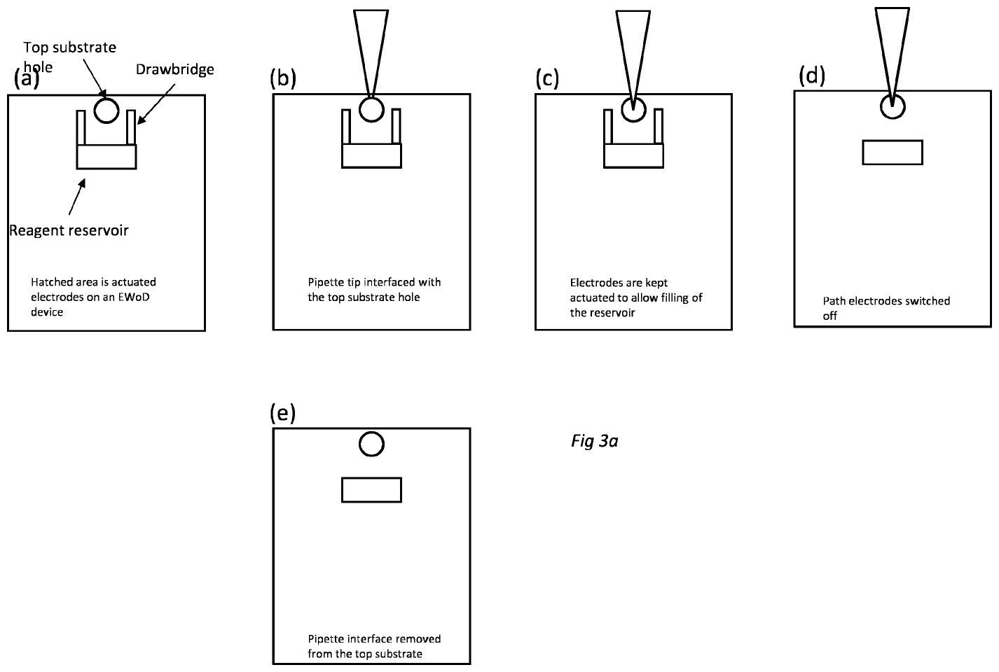

FIG. 3: Schematic showing sequence of actuation pattern for fluid delivery into an EWoD based microfluidic device using paths. The width of the paths is significantly narrow and can cause the breaking of the path. Once the fluid delivery is confirmed, the paths are switched off to prevent the backflow out of the cell gap. The hatched part on the images show the actuation pattern on an EWoD device. FIG. 3a shows the actuation pattern. FIG. 3b represents the liquid flow.

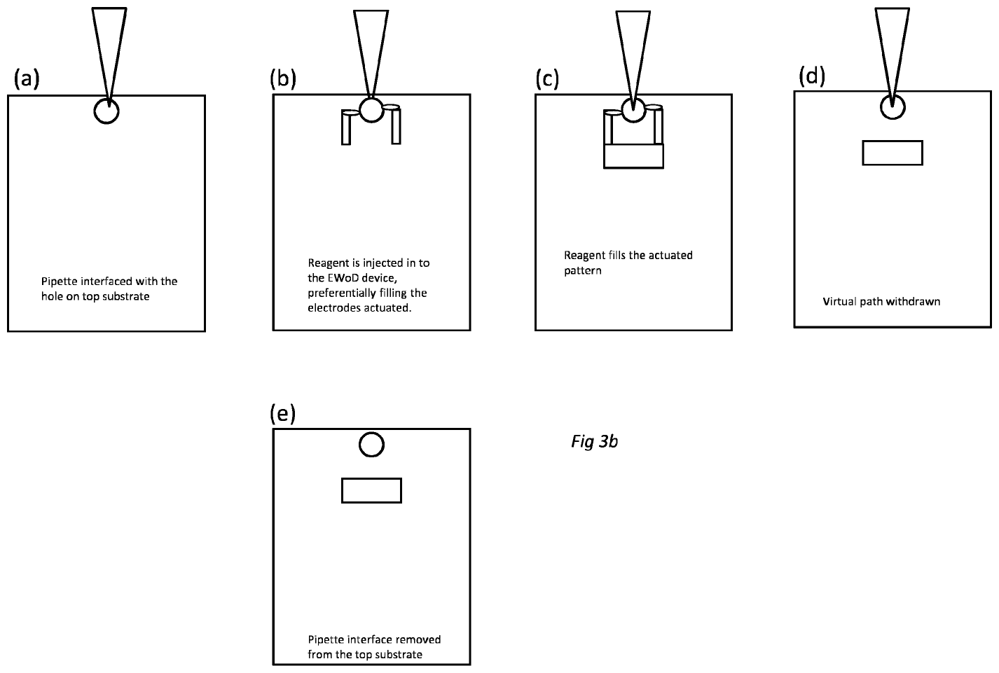

FIG. 4: shows an actuation pattern for the electrodes to deliver liquid into the cell gap using a double path method. The patterns actuated on the both sides of the loading hole collect the liquid to be delivered to the main reservoir and prevent the backflow of the fluid out of the cell gap when the pipette is disconnected. The length and the width of the drawbridge is dependent on the reagent properties and volume to be loaded. (R) shows sequence of images for loading 6.5 μL of aqueous fluid into a base fluid of DMPS with 0.1% Span85. (a) Pipette tip interfaced with a loading port, (b) The aqueous fluid injected into the cell gap and attracted by actuated pixels within the virtual paths, (c-d) the aqueous phase is pushed forward and the main reservoir is filled (in this example the reservoir is 30×30 pixels), (e-g) the pipette is disengaged from the port which results in backflow and break off of the connection between the port and drawbridges, (h) the reservoir is loaded, (i-j) the drawbridges are merged with the reservoir which is resized accordingly.

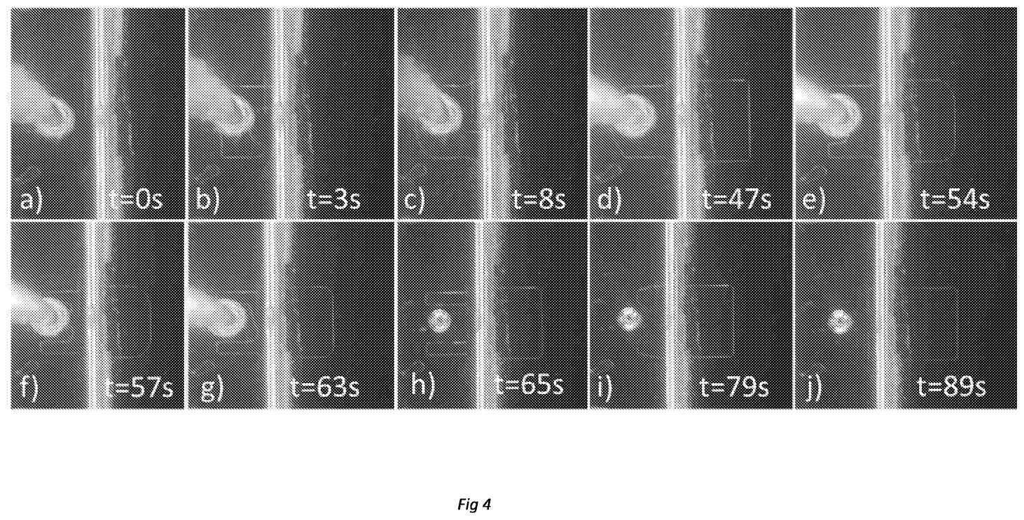

FIG. 5: Image showing reagent loading into a reservoir using a virtual path. Once the pipette is drawn out, the capillary rise of aqueous reagent into the polymer pulls the reagent front leading to necking and breaking the connection.

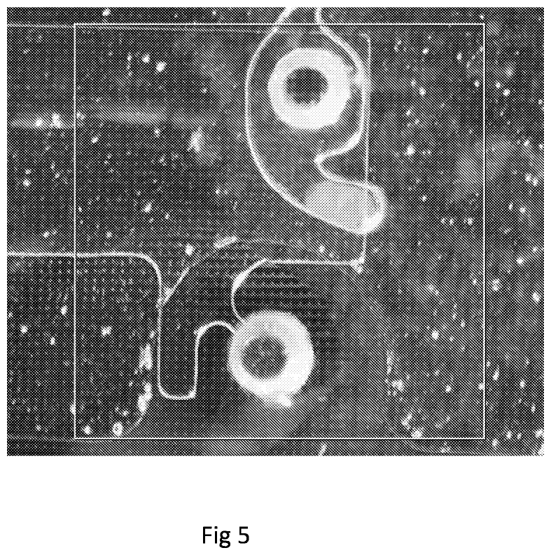

FIG. 6: Image sequence showing loading and formation of a reservoir using virtual channels. The circular inlet port can be used to generate an on-chip reservoir (rectangle) using a narrow virtual path (bridge). When the required volume is loaded, the virtual path is removed to disconnect the reservoir from inlet port. The internal reservoir can then be used to generate droplets.

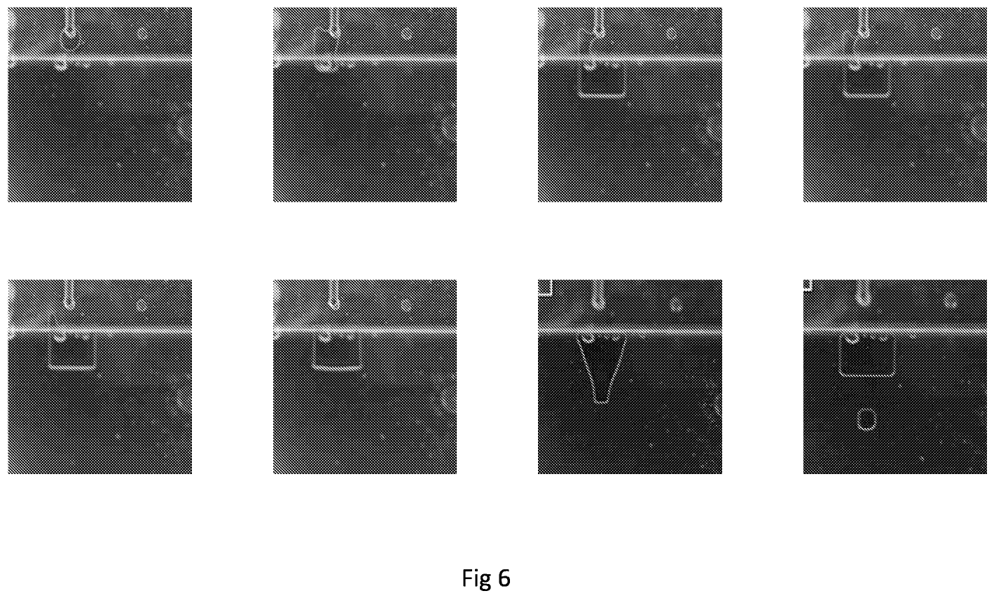

FIG. 7: Image shows an electrode actuation scheme for forming reservoirs with the entry paths activated during fluid entry.

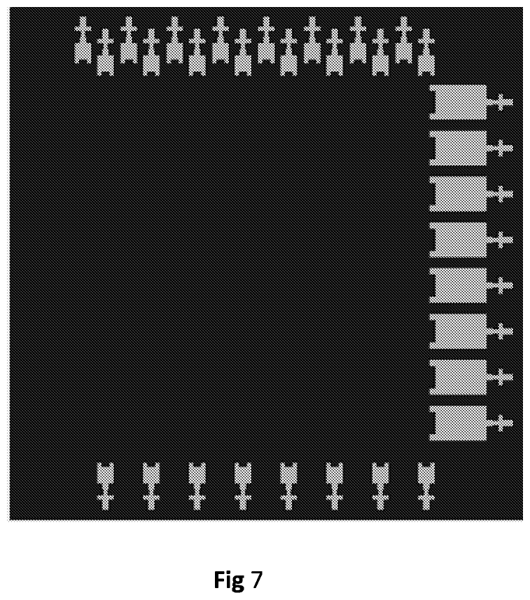

FIG. 8: Image shows an electrode actuation scheme for forming reservoirs with the entry paths switched off after fluid entry.

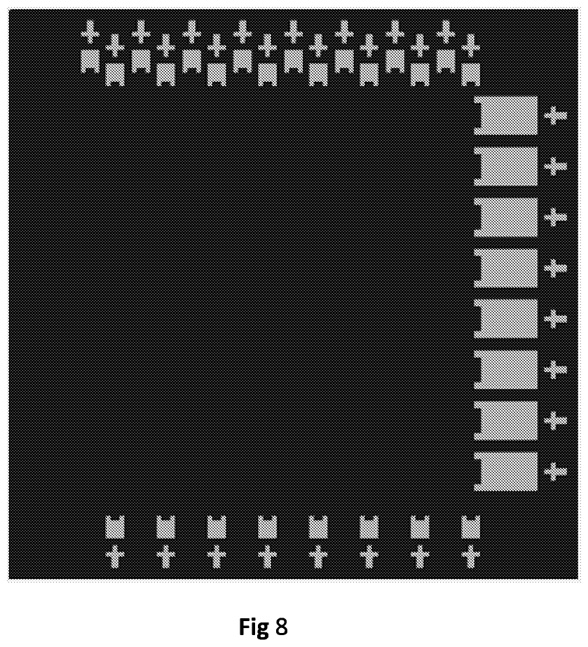

FIG. 9: Images showing actuation pattern for the electrodes to deliver liquid into the cell gap using virtual paths. The red box shows the entry path which allows reliable loading of liquid as shown by green arrow. The square pattern shown by the red arrow in (1) is turned OFF alternatingly (2) during the loading cycle, which together with the other electrodes in the drawbridge prevents the back flow of the fluid out of the cell gap. The length and the width of the drawbridge is dependent on the reagent properties and volume to be loaded. (R) shows sequence of images for loading 2 μL of aqueous fluid in a base fluid of DMPS with 0.1% Span85. (a) Pipette tip interfaced with a loading port, (b) the drawbridge before a pulsing section is filled with aqueous phase, (c) once the pulsing section of drawbridge is filled, the reservoir loading is initiated, (d) the reservoir of defined area is being filled (in this example the reservoir is 16×16 pixels), (e) the loading is completed which is indicated by appearing markers on the opposing side to the entry path, (f) pipette is disengaged from the port which results in backflow and the drawbridge pinches off, (g) the drawbridge pinches off and the loaded reservoir separates from the port (h) the reservoir is loaded

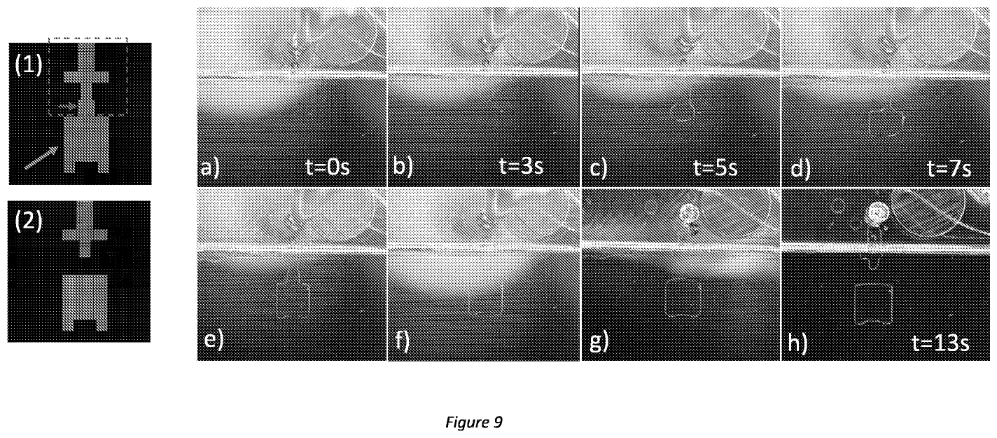

FIG. 10: Image of liquid being loaded in parallel into multiple reservoirs. Narrow necks can be seen between the entry ports and the reservoirs being loaded.

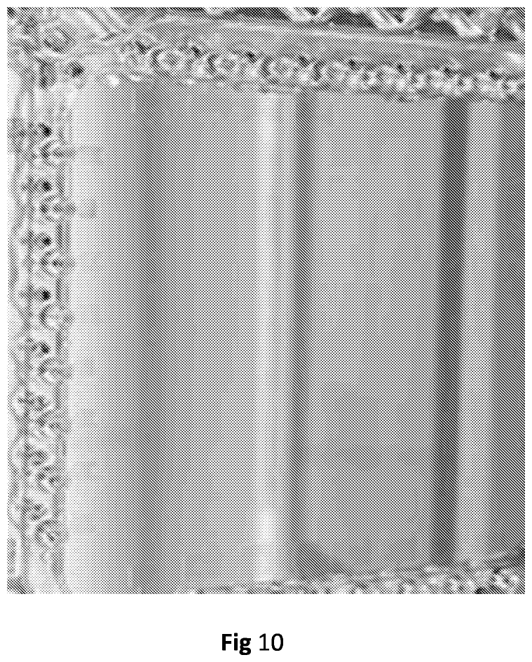

FIG. 11: shows a sequence of images (1-3) demonstrating formation of eight aqueous reservoirs with calibration structures, driven with an air displacement multichannel pipette. The arrow shows the formation of calibration structures on the reservoirs. The volume of the aqueous phase loaded is 5 μL, including both the reservoir to be formed and the calibration structures. The sizes of the actuated areas are 30×28 pixels for the main reservoir and 6×6 pixels for each of the calibration structures. The time required to fill the reservoirs was 120 seconds. The aqueous reagent loaded is 0.05% w/w Pluronic F127 in an aqueous buffer with red food colouring to aid visualisation (1:1 dilution). The filler fluid in the device is 0.1% span85 in dodecamethylpentasiloxane (DMPS). Image (4) shows a snapshot of the electrical actuation pattern sent to the electrodes on the device during reservoir filling, where white represents electrodes with a potential applied. The calibration structures are shown by the arrow on the image. FIG. 4 image (1) shows a DMF device primed with filler fluid. FIG. 4 image (2) shows the initial stages of reservoir loading. FIG. 4 image (3) shows two reservoirs filled to the correct volume (both calibration structures visible) while other reservoirs are still in the process of forming on the device.

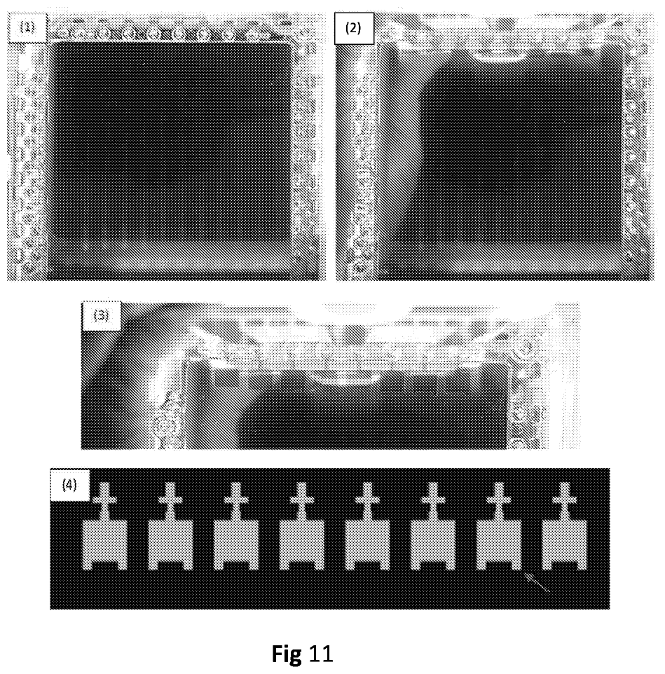

### DETAILED DESCRIPTION

Described herein is a method for loading aqueous reagents into electrowetting devices which are often hydrophobic and therefore problematic to load. In electrowetting devices with plastic housing, there is a net force that results in back flow of fluid after being loaded. The net force comes from the balance of electrowetting force generated with EWoD, Laplace pressure of injected aqueous phase and capillary effect which occurs in the fluid delivery housing. Therefore, the presented method is especially beneficial for loading reagents with larger surface tension and for using plastics to design the fluid delivery housing which are usually hydrophilic. Disclosed is a method of loading which creates a temporary flow path via electrode activation. Activation of electrodes allows liquid entry to a part of the device physically removed from the inlet port. Thus the liquid cannot be ejected from the inlet port once the inlet reservoir or application pressure is removed. The actuation prevents bubble entry to the device. Herein is described a method based on a programmable drawbridge to circumvent issues of poor reagent loading. The drawbridge is a virtual path of electrodes which is actuated to increase the wettability of the EWoD cell gap. Once the required volume has been metered, the drawbridge is withdrawn, i.e. the electrodes deactivated or the inlet source liquid removed, to physically disconnect the reservoir from the fluid applicator inlet port.

The method loads the reservoir using a narrow neck of fluid that can be snapped when a portion of the electrodes in the entry path are switched off. Once the fluid neck in the path is snapped, the liquid can no longer flow towards the inlet port. Many reservoirs can be loaded in parallel from many entry ports. The entry path may comprise a path of varying widths along the path, and a subset of the electrodes on the path can be switched off to disconnect the entry path from the inlet port without moving the reservoir away from the inlet port. The inlet path may comprise a perpendicular bar in order to form a cruciform shape. The cruciform shape provides a decreased radius of curvature for the liquid, in order to promote snapping of the liquid when the path electrodes are switched off. Efficient snapping of liquid may be obtained by pulsing the electrodes, such that they turn off, on and off again, optionally repeatedly. The repeated pulsing allows the neck to narrow before snapping, thereby accurately controlling the liquid volume in each reservoir.

The method is suitable for loading an electrokinetic device including a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes. The method further comprises disposing an aqueous droplet on a first matrix electrode; and providing a differential electrical potential between the first matrix electrode and a second matrix electrode with the voltage source, thereby moving the aqueous droplet.

The method and device can be used when the ionic strength is over 0.1 M and over 1.0 M. The ability to accurately and quickly load high ionic strength solutions offers great utility to those wishing to conduct certain biochemical processes and experiments. High ionic strength solutions are commonly used as wash buffers to disrupt the interaction of nucleic acids and proteins, for example in the commonly performed chromatin immunoprecipitation (ChIP) assay. High ionic strength solutions can also be used for osmotic cell lysis. Additionally, the culture of marine algae is typically performed in media isotonic with seawater, with an ionic strength of 600-700 mM. A further application of high ionic strength solutions is for the elution of proteins from affinity matrices following purification. High ionic strength buffers are also used in enzymatic nucleic acid synthesis. Multiple high ionic strength solutions (1000 mM monovalent or greater) can be used in enzymatic DNA synthesis processes during both washing and deprotection steps.

The dielectric layer may comprise silicon dioxide, silicon oxynitride, silicon nitride, hafnium oxide, yttrium oxide, lanthanum oxide, titanium dioxide, aluminium oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick.

The conformal layer may comprise a parylene, a siloxane, or an epoxy. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The present invention can be used to contact adjacent aqueous droplets by disposing a second aqueous droplet on a third matrix electrode and providing a differential electrical potential between the third matrix electrode and the second matrix electrode with the voltage source.

The invention further provides an assay, nucleic acid synthesis, nucleic acid assembly, nucleic acid amplification, nucleic acid manipulation, next-generation sequencing library preparation, protein synthesis, or cellular manipulation comprising repeating the loading method steps described above.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
a second substrate comprising a top electrode;
a spacer disposed between the first substrate and the second substrate and defining an electrokinetic workspace; and
a voltage source operatively coupled to the matrix electrodes;

The electrokinetic devices as described may be used with other elements, such as for example devices for heating and cooling the device or reagent cartridges for the introduction of reagents as needed.

The devices can be used for any biochemical assay process involving high solute (ionic) strength solutions where the high concentration of ions would otherwise degrade and prevent use of prior art devices. The devices are particularly advantageous for processes involving the synthesis of biomolecules such as for example nucleic acid synthesis, for example using template independent strand extensions, or cell-free protein expression using a population of different nucleic acid templates.

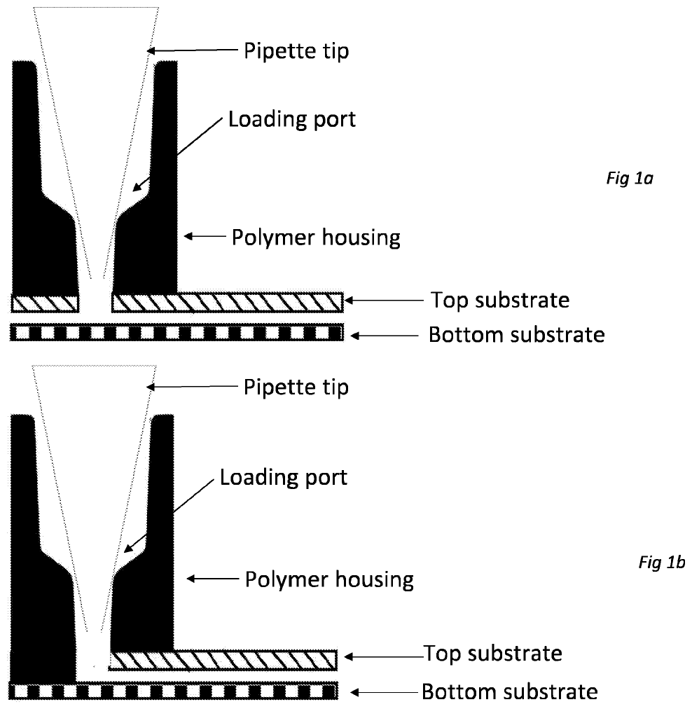

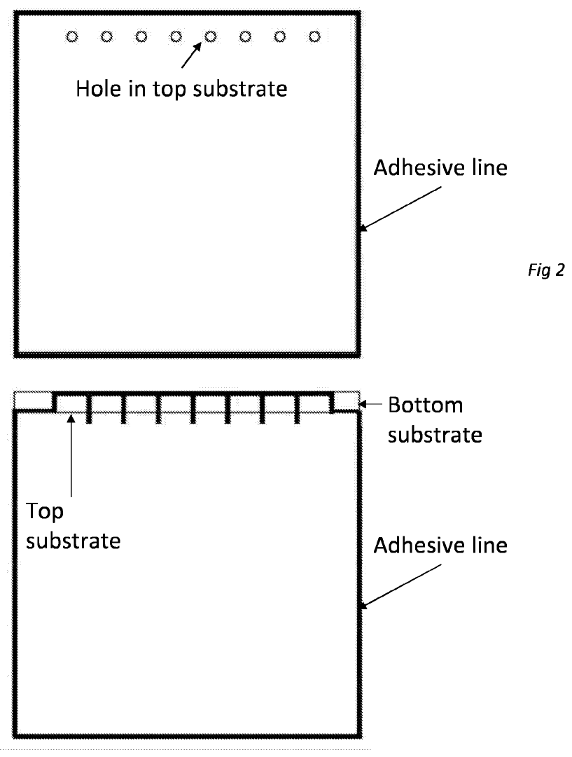

The entry of the liquid can be via the top or side of the array, for example as shown in FIG. 1. FIG. 1a shows a cross sectional view of a EWoD based microfluidic device which has a fluid delivery housing for interfacing to a pipette. The housing interfaces with an aperture in the top substrate. 1b shows the side entry version. Application of liquid via the housing allows liquid into the device. Once the pipette is removed, the liquid is typically ejected as the internal surfaces are hydrophobic and capillary action is not achieved. Liquid entry can however be obtained by activation of specific electrodes to form an entry path onto the device. The hydrophilic activated electrodes allow the liquid to be steered down a defined path. If the electrodes in the vicinity of the inlet are switched off after liquid has been introduced, a ‘necking’ effect is seen and a discreet reagent reservoir is obtained within the device. The reservoir on the device is physically isolated from the entry port, so cannot be ejected from the inlet.

### Applications of the Invention

The invention can be used in a myriad of different applications. In these applications the steps of disposing an aqueous droplet having an ionic strength on a first matrix electrode and providing a differential electrical potential may be repeated many times. They may be repeated over 1000 times or over 10,000 times, sometimes over a 24 hour period.

### Enzymatic DNA Synthesis Applications

The present method can be used in the synthesis of nucleic acids, such as phosphoramidite-based nucleic acid synthesis, templated or non-templated enzymatic nucleic acid synthesis, or more specifically, terminal deoxynucleotidyl transferase (TdT) mediated addition of 3′-O-reversibly terminated nucleoside 5′-triphosphates to the 3′-end of 5′-immobilized nucleic acids. During enzymatic nucleic acid synthesis, the following steps are taken on the instrument:
I. Addition solution containing TdT, optionally pyrophosphatase (PPiase), 3′-O-reversibly terminated dNTPs, and required buffer (including salts and necessary reaction components such as metal divalents) is brought to a reaction zone containing an immobilized nucleic acid, where the nucleic acid is immobilized on a surface such as through magnetic beads via a covalent linkage to the 5′ terminus of the nucleic acid. The initial immobilized nucleic acid may be known as an initiator oligonucleotides and comprises N nucleotides, for example 3-100 nucleotides, preferably 10-80 nucleotides, and more preferably 20-65 nucleotides. Initiator oligonucleotides may contain a cleavage site, such as a restriction site or a non-canonical DNA base such as U or 8-oxoG. Addition solution may optionally contain a phosphate sensor, such as E. coli phosphate-binding protein conjugated to MDCC fluorophore, to assess the quality of nucleic acid synthesis as a fluorescent output. dNTPs can be combined in ratios to make DNA libraries, such as NNK syntheses.
II. Wash solution, either in bulk or in discrete droplets, is applied to reaction zones to wash away the addition solution. Wash solution typically has a high solute concentration (>1 M NaCl).
III. Deprotection solution, either in bulk or in discrete droplets, is applied to reaction zones to deprotect the 3′-O-reversible terminator added to the immobilized nucleic acids in the immobilized nucleic acid zone in step I. Deprotection solution typically has a high solute concentration.
IV. Wash solution, either in bulk or in discrete droplets, is applied to reaction zones to wash away the deprotection solution.
V. Steps I-IV are repeated until desired sequences are synthesized, for example steps I-IV are repeated 10, 50, 100, 200 or 1000 times.

The present method can be used in the preparation of oligonucleotide sequences, either via synthesis or assembly. The device allows synthesis and movement of defined sequences. Using the present method the initiation sequences can be modified at a specific location above an electrode and the extended oligonucleotides prepared. The initiation sequences at different locations can be exposed to different nucleotides, thereby synthesising different sequences in different regions of the electrowetting device.

After synthesis of a defined population of different sequences in different regions of the electrowetting device, the sequences can be further assembled in longer contiguous sequences by joining two or more synthesised strands together.

Described herein is a method for preparing a contiguous oligonucleotide sequence of at least 2n bases in length comprising taking the electrowetting device as described herein having a plurality of immobilised initiation oligonucleotide sequences, one or more of which contains a cleavage site, using the initiation oligonucleotide sequences to synthesise a plurality of immobilised oligonucleotide sequences of at least n bases in length, using cycles of extension of reversibly blocked nucleotide monomers, selectively cleaving at least two of the immobilised oligonucleotide sequences of least n bases in length into a reaction solution whilst leaving one or more of the immobilised oligonucleotide sequences attached, hybridizing at least two of the cleaved oligonucleotides to each other, to form a splint, and hybridizing one end of the splint to one of the immobilized oligonucleotide sequences and joining at least one of the cleaved oligonucleotides to the immobilised oligonucleotide sequences, thereby preparing a contiguous oligonucleotide sequence of at least 2n bases in length.

The present invention can be used to automate the movements of droplets in a cartridge. For example, droplets intended for analysis can be moved according to the present invention. The present invention could be incorporated into a cartridge used for local clinician diagnostics. For example it could be used in conjunction with nucleic acid amplification testing (NAAT) to determine nucleic acid targets in, for example, genetic testing for indications such as cancer biomarkers, pathogen testing for example detecting bacteria in a blood sample or virus detection, such as a coronavirus, e.g. SARS-COV-2 for the diagnosis of COVID-19.

The device may be thermocycled to enable nucleic acid amplification, or the device may be held at a desired temperature for isothermal amplification. Having different sequences synthesised in different regions of the device allows multiplex amplification using different primers in different regions of the device.

Furthermore the invention can be used in conjunction with next generation sequencing in which DNA is synthesised by the addition of nucleotides and large numbers of samples are sequenced in parallel. The present invention can be used to accurately locate the individual samples used in next generation sequencing.

The invention can be used to automate library preparation for next generation sequencing. For example the steps of ligation of sequencing adaptors can be carried out on the device. Amplification of a selective subset of sequences from a sample can then have adaptors attached to enable sequencing of the amplified population.

### Protein Expression Applications

The method of moving aqueous droplets may also be used to help facilitate cell-free expression of peptides or proteins. In particular, droplets containing a nucleic acid template and a cell-free system having components for protein expression in an oil-filled environment can be moved using a method of the invention in the described electrokinetic device.

Disclosed herein is a method for the real-time monitoring of in vitro protein synthesis comprising
a. in vitro transcription and translation of a protein of interest fused to a peptide tag; and
b. monitoring the presence of the peptide tag using a further polypeptide which in the presence of the peptide tag produces a detectable signal.

Disclosed herein is a method for the monitoring of cell free protein synthesis in a droplet on a digital microfluidic device comprising
a. cell free transcription and translation of a protein of interest fused to a peptide tag; and
b. monitoring the presence of the peptide tag using a further polypeptide which in the presence of the peptide tag produces a detectable signal.

The use of the terms “in-vitro” and “cell free” may be used interchangeably herein.

The detectable signal may be for example fluorescence or luminescence. The detectable signal may also be caused by the binding of a ligand to the complemented oligopeptide, peptide, or polypeptide tag fused to the protein of interest.

The detectable signal may also be caused by the binding of the polypeptide to the protein of interest fused to a His-tag.

Any in vitro transcription and translation may be used, for example extract-based systems derived from rabbit reticulocyte lysate, human lysate, Chinese Hamster Ovary lysate, a wheat germ, HEK293 lysate, E. coli lysate or yeast lysate.

Alternatively the in vitro transcription and translation may be assembled from purified components, for example a system of purified recombinant elements (PURE).

The in vitro transcription and translation may be coupled or uncoupled.

The peptide tag may be one component of a fluorescent protein and the further polypeptide a complementary portion of the fluorescent protein. The fluorescent protein could include sfGFP, GFP, ccGFP, eGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide NFAST in the presence of a hydroxybenzylidene rhodanine analog.

The peptide tag may also be one component of a protein that forms a detectable substrate, such as a luminescent or colorigenic substrate. The protein could include beta-galactosidase, beta-lactamase, or luciferase.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

Any protein of interest may be synthesised. The protein may be an enzyme, for example a terminal deoxynucleotidyl transferase (TdT) enzyme or a truncated version thereof or the homologous amino acid sequence of a terminal deoxynucleotidyl transferase (TdT) enzyme in other species or the homologous amino acid sequence of Polμ, Polβ, Polλ, and Polθ of any species or the homologous amino acid sequence of X family polymerases of any species.

Protein expression typically requires an ample supply of oxygen. The most convenient and high yielding way to power CFPS is via oxidative phosphorylation where O₂ serves as the final electron acceptor; however, there are other ways that involve replenishing with energy molecules not involved in oxidative phosphorylation. In a confined microfluidic or digital microfluidic system of droplets, insufficient oxygen is available to enable efficient protein synthesis.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrowetting. The components for the cell-free protein synthesis droplet can be pre-mixed prior to introduction to or mixed on the digital microfluidic device.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static.

The droplet can be moved using any means of electrowetting. The droplet can be moved using electrowetting-on-dielectric (EWoD). The electrical signal on the EWoD or optical EWoD device can be delivered through segmented electrodes, active-matrix thin-film transistors, or digital micromirrors.

The filler fluid in the device can be any water immiscible liquid. The filler fluid can be mineral oil, silicone oil such as dodecamethylpentasiloxane (DMPS), an alkyl-based solvent such as decane or dodecane, or a fluorinated oil. The filler fluid can be oxygenated prior to or during the expression process.

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with additive droplets for screening.

Included herein is a method wherein multiple reservoirs are formed in parallel. The reservoirs can contain different reagents. For example a subset of the reservoirs may contain nucleic acid templates and a subset contain a cell-free system having components for protein expression. The reservoirs can be split and merged with the components from other reservoirs in order to initiate reactions.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk filler liquid.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

The filler fluid in the device can be any water immiscible, non-ionic or hydrophobic liquid. The oil can be mineral oil, silicone oil such as dodecamethylpentasiloxane (DMPS), an alkyl-based solvent such as decane or dodecane, or a fluorinated oil.

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

Through an affinity tag, such as a FLAG-tag, HIS-tag, GST-tag, MBP-tag, STREP-tag, or other form of affinity tag, CFPS-expressed proteins can be immobilized to a solid-support affinity resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF- and CE-CFPS, users can scale up their CFPS production methods.

Droplets can also contain additives to reduce the effects of biofouling on digital microfluidic surfaces. Specifically, droplets containing CFPS components can also contain additives such as surfactants or detergents to reduce the effects of biofouling on the hydrophobic or superhydrophobic surface of a digital microfluidic device (Langmuir 2011, 27, 13, 8586-8594). Such droplets may use antifouling additives such as TWEEN 20, Triton X-100, and/or Pluronic F127. Specifically, droplets containing CFPS components may contain TWEEN 20 at 0.1% v/v, Triton X-100 at 0.1% v/v, and/or Pluronic F127 at 0.08% w/v.

Rather than adding surfactants to the aqueous sample, it is instead possible to add surfactant, such as a sorbitan ester such as Span85 (e.g. Sorbitan trioleate, Sigma Aldrich, SKU 8401240025), to the filler liquid. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be non-ionic, anionic, cationic, amphoteric or a mixture thereof. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the Split GFP (e.g. GFP₁₁/GFP₁₋₁₀) system, so removing surfactants from the reagent mix and instead adding them to the oil can be beneficial.

The peptide tag can be attached to the C or N terminus of the protein. The peptide tag may be one component of a green fluorescent protein (GFP). For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀.

The protein may be fused to multiple tags. For example the protein may be fused to multiple GFP₁₁ peptide tags and the synthesis occurs in the presence of multiple GFP₁₋₁₀ polypeptides.

For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

Where used herein “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A method for loading aqueous liquids from an external source into a planar EWoD device having an array of electrodes, the method comprising;
a. taking an EWoD device having an inlet port containing an aqueous liquid,
b. actuating reservoir electrodes to form a defined reservoir of aqueous liquid on the device wherein the defined reservoir is separated from the inlet port by at least two electrodes so as not to overlap the inlet port;
c. actuating specific path electrodes on the device from the inlet port to form a virtual path for aqueous liquid entry over the electrodes onto the device, wherein the virtual path is narrower than the reservoir; and
d. switching off at least two of the electrodes in the virtual path to separate the reservoir from the inlet port, thereby preventing back-flow of the aqueous liquid from the reservoir to the inlet port.

2. The method according to claim 1 wherein the electrode activation pattern defines the volume of liquid held in the reservoir.

3. The method according to claim 1 or claim 2 wherein the number of electrodes activated to form the width of the virtual path is less than half the number forming the width of the defined reservoir.

4. The method according to claim 3 wherein the number of electrodes activated to form the width of the virtual path is less than one quarter the number forming the width of the defined reservoir.

5. The method according to any one of claims 1 to 4 wherein multiple virtual paths connect the inlet to the reservoir.

6. The method according to any one of claims 1 to 5 wherein the virtual path comprises a cruciform shape.

7. The method according to any one of claims 1 to 6 wherein the virtual path comprises four sections of different widths, at least one of which is switched off to separate the reservoir from the inlet port.

8. The method according to any one of claims 1 to 7 wherein electrodes in the virtual path are pulsed off and on and off.

9. The method according to any one preceding claim wherein the inlet port comprises a hole in the surface of the planar EWoD device.

10. The method according to any one of claims 1 to 7 wherein the inlet port comprises a hole in the side of the planar EWoD device.

11. The method according to any one preceding claim wherein the array of electrodes are formed on the surface of the planar EWoD device opposing the inlet port.

12. The method according to any one preceding claim wherein the aqueous liquid in the inlet port is loaded from an external source in the form of a pipette, multichannel pipette or delivery tube.

13. The method according to any one preceding claim wherein the electrode actuation to form the virtual path occurs for a period of greater than 1 second.

14. The method according to any one preceding claim wherein the delivery path is formed by actuating between 10-500 electrodes arranged in an elongated pattern.

15. The method according to claim 14 wherein the delivery path is formed by actuating electrodes arranged in an elongated pattern of 22 to 35 electrodes long by 4 to 8 electrodes wide.

16. The method according to any one preceding claim wherein the on-chip reservoir is formed 20-100 electrodes away from the inlet port.

17. The method according to any one preceding claim wherein the on-chip reservoir is 0.1 to 100 μL.

18. The method according to any one preceding claim wherein multiple on-chip reservoirs are formed using a single inlet port by actuating different virtual paths.

19. The method according to any one preceding claim wherein multiple inlet ports and virtual paths are used to combine reagents into one or more on-chip reservoirs.

20. The method according to any one preceding claim wherein multiple reservoirs are formed in parallel from multiple inlet ports.

21. The method according to any one preceding claim wherein the method comprises temporarily actuating electrodes on an opposing side of the reservoir to the source liquid to form one or more virtual calibration structures which are the last areas to fill, such that when the temporarily actuated electrodes are switched off the liquid becomes part of the reservoir, thereby accurately controlling the liquid area in the reservoir.

22. The method according to claim 21 wherein the virtual calibration structures are elongated protrusions and there are two or three elongated protrusions per reservoir.

23. The method according to any one preceding claim comprising
a. taking an EWoD device having an inlet port containing an aqueous liquid,
b. actuating reservoir electrodes to form a defined reservoir of aqueous liquid on the device wherein the defined reservoir is separated from the inlet port by at least two electrodes so as not to overlap the inlet port and the reservoir includes electrodes on an opposing side of the reservoir to the source liquid to form one or more virtual calibration structures which are the last areas to fill;
c. actuating specific path electrodes on the device from the inlet port to form a virtual path for aqueous liquid entry over the electrodes onto the device, wherein the virtual path is narrower than the reservoir and forms a cruciform shape;
d. switching off at least two of the electrodes in the virtual path to separate the reservoir from remaining cruciform shape and hence the inlet port, thereby preventing back-flow of the aqueous liquid from the reservoir to the inlet port; and
e. switching off the virtual calibration structures.

24. The method according to any one preceding claim wherein the EWoD device includes:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
a second substrate comprising a top electrode;
a spacer disposed between the first substrate and the second substrate and defining an electrokinetic workspace; and
a voltage source operatively coupled to the matrix electrodes.

25. The method according to any one preceding claim wherein the aqueous liquid has an ionic strength greater than 0.01 M.

26. A method according to claim 20, wherein a subset of the reservoirs contain nucleic acid templates and a subset contain a cell-free system having components for protein expression.
